(19) United States
(12) Patent Application Publication
(10) Pub. No.: US 2015/0086657 A1
(43) Pub. Date: Mar. 26, 2015

(54) EUCOMMIA ULMOIDES-CONTAINING CHOCOLATE COMPOSITIONS

(71) Applicant: Platinum Naturals Ltd., Richmond Hill (CA)

(72) Inventor: David Tae-Won Khang, North York (CA)

(73) Assignee: Platinum Naturals Ltd., Richmond Hill (CA)

(21) Appl. No.: 14/493,531

(22) Filed: Sep. 23, 2014

Related U.S. Application Data
(60) Provisional application No. 61/881,521, filed on Sep. 24, 2013.

(51) Int. Cl. A23G 1/42; A61K 36/46; A23G 1/48; A61K 36/48

(52) U.S. Cl. CPC A23G 1/42; A61K 36/48; A61K 36/46; A23G 1/48
USPC 424/757

(57) ABSTRACT

The present invention generally relates to edible compositions containing at least one plant extract with estrogenic properties including, for example, those exhibiting properties suitable for treatment of conditions or symptoms associated with menopause. In particular, the present invention generally relates to such edible compositions containing eucommia ulmoides (optionally in combination with a mung bean extract or composition). The present invention also particularly relates to such compositions in the form of an edible chocolate composition containing eucommia ulmoides (optionally in combination with a mung bean extract or composition).

### REFERENCE TO RELATED APPLICATION

This application claims priority to provisional application U.S. Ser. No. 61/881,521, filed Sep. 24, 2013, the entire disclosure of which is incorporated herein by reference.

### FIELD OF THE INVENTION

The present invention generally relates to edible compositions containing at least one plant extract with estrogenic properties including, for example, those exhibiting properties suitable for treatment of conditions or symptoms associated with menopause. In particular, the present invention generally relates to such edible compositions containing eucommia ulmoides (optionally in combination with a mung bean extract or composition). The present invention also particularly relates to such compositions in the form of an edible chocolate composition containing eucommia ulmoides (optionally in combination with a mung bean extract or composition).

### BACKGROUND OF THE INVENTION

Herbal formulations comprising extracts of one or more herbal plants have been used for centuries in Traditional Chinese Medicine (TCM). Eucommia Ulmoides (EU) is a large deciduous tree which originated in China. The bark of the tree (commonly referred to as Cortex eucommiae) has been known for use in natural medicine for some time. Compositions based on or derived from EU bark have been used for, among other things, the relief of back pain, to increase strength, to strengthen bones and muscle, to improve recovery from fatigue, to increase ability to remember and to induce an anti-aging effect. Compositions based on or derived from EU leaves are also suitable for use. Advantageously, ingestion of EU bark and leaves, and/or their extracts do not cause any known side effects.

Most all women at some point deal with at least one estrogenic-mediated condition, including those associated with hypoestrogenic states such as menopause. There are numerous known treatments for use in connection with menopause-related symptoms and conditions. However, there exists a need in the art for improved compositions and methods suitable for treatment of menopause-related symptoms and conditions.

### SUMMARY OF THE INVENTION

Briefly, therefore, the present invention is directed to compositions comprising at least one pharmaceutically or nutraceuticlally active agent, specifically the combination of a eucommia ulmoides (EU) composition and a mung bean extract or composition. The present invention is particularly directed to edible chocolate compositions comprising at least one pharmaceutically or nutraceutically active agent, the composition comprising a chocolate base, a eucommia ulmoides (EU) composition, and a mung bean extract or composition. The EU composition and the mung bean composition are dispersed throughout the chocolate base.

The present invention is also directed to methods of treatment of one or more menopause-related symptoms or conditions, the method comprising administering to a subject in need thereof a composition of the present invention.

Other objects and features will be in part apparent and in part pointed out hereinafter.

### DETAILED DESCRIPTION OF THE PREFERRED EMBODIMENTS

In accordance with the present invention, it has been discovered that chocolate-based compositions comprising an EU product (and optionally a mung bean extract) are suitable for use in treatment of symptoms or conditions associated with menopause. In particular, it is currently believed that the present compositions are suitable for treating or combating vasomotor symptoms and side effects of declining estrogen levels in menopausal women. More particularly, it has been discovered that such compositions may provide one or more improvements over conventional compositions for treatment perimenopause and menopause related symptoms or conditions. For example, such compositions are believed to provide improvements in patient compliance and easier digestion. It is further currently believed that the chocolate-based compositions of the present invention provide improved active ingredient stability as compared to other compositions including EU and one or more other plant extracts including, for example, capsule-based compositions.

Generally, the compositions of the present invention include EU (or an extract thereof) dispersed throughout a chocolate-based carrier, or composition. The compositions may also include other ingredients including, for example, a mung bean component or extract.

### Eucommia Ulmoides (EU)

Suitable EU compositions or extracts are generally obtained from an EU-containing plant, or portion of the plant, including from the leaves or the bark of a tree. The EU composition or extract generally contains one or more active components considered to have estrogenic properties (e.g., act as an “estrogen modulator”) which regulate female hormone function. In this manner, as noted the EU compositions are effective for use in compositions for treatment of symptoms or conditions associated with menopause. Compounds identified as present in EU extracts derived from bark include various lignan diglucosides, pinoresinol, glucopyranoside, syringaresinol, gutta-percha (6-10%), glucans (0.26%), eucommioside, eucommiol, geniposide, and/or ulmoprenol. Compounds identified as present in EU extracts derived from leaves include caffeic acid, chlorogenic acid, ferulic acid, quercetin, rutin, isoquercitrin, ulmoidol, corosolic acid, oleanolic acid, ursolic acid, foliasalacioside, and/or quercetin. One or more of these compounds may be present in the EU extract and/or may be an active compound of EU extract. However, activity of any or all of these compounds is not required in accordance with the present invention, but the presence of one or more of these compounds may nonetheless be identified in a composition and therefore identify the presence of an EU extract.

An EU composition utilized in accordance with the present invention is typically provided in the form of an EU extract derived from one or more EU-containing portions of the plant. Generally, an EU extract is provided by grinding, or macerating the tissues of the plant, extracting the active EU compounds using a solvent system, and separating the liquid phase containing active EU compounds from the solid phase. In addition to active EU compounds, there are often undesired compounds that may cause undesired side-effects or side-reactions and/or reduce efficacy of the active EU compounds. These compounds may be removed from the liquid fraction by precipitation caused by addition of water to the liquid phase.

In accordance with the present description, it is to be understood that the term “EU extract” may refer to a liquid fraction derived from an EU-containing source containing one or more active EU compounds. “EU extract” may also refer to a dried extract obtained during the solvent extraction process, which may be utilized in dry form for formulating a composition, or may be diluted prior to use to form a liquid EU extract. Typically, dried extracts are utilized in preparing compositions of the present invention.

EU compositions or extracts may be obtained from the leaves or bark of an EU-containing plant or tree, but in various preferred embodiments EU compositions are derived from the bark of an EU-containing tree.

In a solvent-based extraction method, typically the ground or macerated plant portion is soaked with a solvent system and left for a period of time to allow the active compounds to dissolve into the solvent system. To enhance the diffusion of the active compounds into the solvent system, the mixture can be mechanically agitated and/or heated to a pre-determined temperature. Suitable mechanical agitation methods include but are not limited to shaking, vortexing, swirling, stirring, and ultrasonicating. After a sufficient period of time for the diffusion of the active compounds into the solvent system, the liquid is separated from the solid by any one of the well-known techniques such as filtration and centrifugation.

The solvent system can comprise any of the well-known systems such as an organic solvent or a combination of organic solvents. Typically, the organic solvent is an alcohol and, more typically, ethanol. Other organic solvents such as hexane, dichloromethane, ethyl acetate are also suitable.

Water is also typically part of the solvent system, with the proportion of water broadly ranging from 5 wt % to 95 wt %. If water is not initially present as part of the solvent system, it can be added to the liquid phase during any part of the soaking period. It is also possible to add water to the liquid phase after separation from the solid phase. As noted above, one of the effects of the addition of water is to cause precipitation of non-active EU compounds from the solvent system that may have adverse side-effects. Water may be added in a proportion of up to 20% by volume of the solvent system.

Generally in accordance with the present invention, the solvent extraction system may include ethanol and water. The proportion of water may range from 5 to 95 wt %, from 20 to 80 wt %, or from 30 to 70 wt %. Similarly, the proportion of alcohol (e.g., ethanol) may range from 5 to 95 wt %, from 20 to 80 wt %, or from 30 to 70 wt %. In various preferred embodiments, the solvent system comprises water in a proportion of from 20 to 95 wt %, and ethanol in a proportion of from 5 to 80 wt %. Thus, in certain embodiments, the solvent system is a (95:5) (wt:wt) water:ethanol solvent system, or a (20:80) (wt:wt) water:ethanol solvent system.

Another method of extracting active compounds from the macerated EU plant is to percolate the macerated EU plant with a continuously refluxed solvent system such as the soxlet-type method for extraction. After completion of the extraction process, the liquid containing the active compounds may be mixed with water (up to 20% by volume) to precipitate the undesired compounds. The precipitate can be separated from the liquid containing the active EU compounds by, for example, filtration and/or centrifugation.

Regardless of the precise method of providing the EU extract, a liquid phase including active EU compounds and at least a portion of the solvent system is provided. The solvent portion of this liquid phase containing active EU compounds can be evaporated using any suitable drying method known in the art including, for example, rotary evaporation, speed-vacuum centrifugation or simply drying (e.g., oven-top drying). The dried extract is then suitable for use or storage. However, as noted above, if desirable the dried extract can be diluted or re-suspended in a suitable solvent (e.g., water) prior to use. Typically, dried EU extracts having a solids content of at least about 70 wt %, at least about 80 wt %, or at least about 90 wt % (e.g., at least about 95 wt %) are utilized in the compositions of the present invention.

Once an extract containing active EU compounds is provided, it may be utilized in a composition in accordance with methods generally known in the art and including the discussion herein.

Typically, the particle size of at least a portion of a dried EU extract is 80 mesh or less.

### Mung Beans

In various embodiments, the composition includes a mung (or moong) bean fraction. The mung bean (also known as the green gram or golden gram) is the seed of vigna radiata, is native to the Indian subcontinent, and mainly cultivated in India, China, Thailand, Phillipines, Indonesia, Burma, Bangladesh, Vietnam, Laos, and Cambodia, but also in hot and dry regions of Southern Europe and the Southern United States.

Mung beans have been used for a variety of purposes including, for example, lowering LDL cholesterol levels. For example, mung beans contain approximately 1.9 grams (g) of fiber per 8 oz. and, therefore, are suitable for use either alone or in combination with other fiber-rich foods for use in lowering LDL cholesterol levels. Mung beans also contain protease inhibitors. Protease inhibitors are known to slow the replication of certain cancer cells, therefore mung bean compositions may be used in connection with cancer treatments, including breast cancer treatments. As a low-glycemic index food, mung beans may also be used in treatments in connection with diabetes. Mung beans (and other beans) contain isoflavone nutrients, which are known to regulate hormonal activity. Mung beans generally belong to a group of plants known as phytoestrogens (i.e., natural, plant-based estrogens). Phytoestrogens are useful in supplementing declining estrogen levels. In accordance with the present invention, mung beans (in combination with active compounds derived from EU) are currently believed to be useful in the present compositions for the treatment of conditions and symptoms associated with perimenopause and menopause. For example, it is currently believed that combining these components with cocoa provides benefits in supporting cognitive function, balance mood fluctuations, and reduce the severity or occurrence of hot flushes, in addition to treating various other conditions discussed elsewhere herein.

A mung bean extract may be prepared by a solvent-based method generally as described above in connection with EU extracts and compositions. Such a method generally includes grinding, or macerating mung beans and extracting using a suitable solvent system. Generally in accordance with the present invention, the solvent extraction system may include ethanol and water. The proportion of water may range from 5 to 95 wt %, from 20 to 80 wt %, or from 30 to 70 wt %. Similarly, the proportion of alcohol (e.g., ethanol) may range from 5 to 95 wt %, from 20 to 80 wt %, or from 30 to 70 wt %. In various preferred embodiments, the solvent system comprises water in a proportion of from 20 to 95 wt %, and ethanol in a proportion of from 5 to 80 wt %. Thus, in certain embodiments, the solvent system is a (95:5) (wt:wt) water:ethanol solvent system, or a (20:80) (wt:wt) water:ethanol solvent system.

Generally, the solvent system is contacted with the mung bean (typically a ground mung bean fraction) at a weight ratio of solvent system to mung bean fraction of at least 2.5:1, at least 5:1, or at least 7.5:1. Typically, this ratio is from about 2.5:1 to about 15:1, from about 5:1 to about 15:1, or from about 7.5:1 to about 12.5:1.

A dried mung bean extract may be obtained as detailed above by removing the solvent portion of the extraction system. Typically, a dried mung bean extract is utilized in preparing compositions of the present invention. In particular, typically a dried mung bean extract having a solids content of at least about 70 wt %, at least about 80 wt %, or at least about 90 wt % (e.g., at least about 95 wt %) are utilized in the compositions of the present invention.

Typically, the particle size of at least a portion of a dried mung bean extract is 80 mesh or less.

### Edible Compositions

Generally, compositions of the present invention include an EU extract and chocolate, and typically further include a mung bean extract. Compositions of the present invention including an EU extract and mung bean dispersed in a chocolate base, or carrier are currently believed to provide various advantages. It is currently believed that chocolate-based compositions provide one or more advantages including, for example, improved patient compliance as subjects are more likely to desire consuming a chocolate-based composition as compared to other oral dosage forms such as capsules or tablets. It is also currently believed that the chocolate-based compositions are more easily digestible as compared to other conventional dosage forms. It is further currently-believed that the chocolate-based composition provides improvements in stability of the EU and mung bean active ingredients based on one or more of the following reasons. Since water is a universal solvent, it has a tendency to adversely affect active ingredient stability. Chocolate has a low water activity, which is thus believed to avoid this adverse effect of water on active ingredient stability. In addition, oxygen and light are known to adversely affect active ingredient stability, but the cocoa powder and sugar components of the chocolate act as oxygen and light barriers. Additionally or alternatively, it is currently believed that the anti-oxidants present in chocolate prevent oxidation and deactivation of the active ingredients. Although one or more of these effects may be provided by the compositions of the present invention it is to be understood that none or all of them are required.

Although chocolate-based compositions to deliver various active ingredients are known, to-date such compositions including an EU extract (optionally including a mung bean extract or composition) have not been developed. As detailed below, it has been discovered that certain processing strategies are employed to provide the chocolate-based EU extract+mung bean compositions.

Chocolate compositions are believed to be desirable at least on the basis of providing an improvement in patient compliance as chocolate compositions are generally more desirable for consumption than, for example, capsules, tablets, or pills. Chocolate has also been reported to be well-suited as a vehicle for delivery of a variety of active agents. For example, chocolate is a substantially anhydrous medium that is resistant to microbial growth and hydrolysis of water-sensitive active agents. Additionally, or alternatively it is currently believed that the bitter taste of chocolate (in particular dark chocolate) stimulates the release of bile and therefore improved absorption of fats, including omega oils, which are needed for hormone production. It is currently believed that improved fat absorption and its potential impact on hormone productions may provide a particular advantage(s) in connection with treatment of estrogen-mediated disorders such as symptoms associated with perimenopause and/or menopause.

Suitable chocolate for use in the compositions of the present invention may be obtained using any of the various processes known in the art and the term “chocolate” includes, without limitation, sweet chocolate, semi-sweet chocolate, dark chocolate, milk chocolate, white chocolate, couverture chocolate, baking chocolate, or any other known in the art. In various preferred embodiments, dark chocolate is used. It has been observed that dark chocolate-based compositions exhibit improved flavor characteristics and, thus, may be preferred. Without being bound to a particular theory, it is currently believed that the bitter taste of dark chocolate may trigger gastric flow and, therefore, result in improved absorption of the EU extract and/or mung bean extract, including active components thereof. However, it is to be noted that although dark chocolate may provide improved flavor and/or active ingredient performance, other chocolates (e.g., milk chocolate) are also suitable for providing a product having an acceptable flavor.

Chocolate processing generally includes the steps of blending, conching, and tempering. During the blending step, chocolate (cocoa) liquor is mixed with other components to provide the desired type of chocolate. For example, in the case of dark chocolate, the chocolate (cocoa) liquor is mixed with sugar, cocoa butter (and optionally vanilla) in proportions suitable to provide the chocolate of the desired composition. For example, dark chocolate typically contains at least 70 wt % cocoa, while milk chocolate typically contains at least 50 wt % cocoa. After blending to form the desired type of chocolate, the next step in the process is conching. Conching includes maintaining the chocolate in a liquid state by frictional heat, for example in a container filled with metal beads that act as grinders. Prior to conching, chocolate has a gritty and uneven texture. Conching produces a chocolate having smooth mouth feel by virtue of providing cocoa and sugar particles smaller than can be felt or detected. Once conching is complete, the chocolate mass is stored in a suitable container under heat (typically from about 45 to about 50° C.) until further processing.

The final process is tempering.

During tempering, the fats in cocoa butter solidify (i.e., crystallize) into one of six polymorphic forms (I-VI), each of which includes a fat matrix (i.e., a three-dimensional arrangement of fat molecules) that is substantially crystalline. Following are the melting ranges of the six crystalline forms of cocoa butter:

| Polymorph | Melting Range (° C.) |
|---|---|
| I | 16-18 |
| II | 22-24 |
| III | 24-26 |
| IV | 26-28 |
| V | 32-34 |
| VI | 34-36 |

Each polymorph is suitable for forming chocolate that may be used in compositions of the present invention. However, advantageously at least a significant fraction (and preferably nearly all) of the chocolate is present as Polymorph V. This form is solid at room temperature, but advantageously melts easily at typical mouth temperatures and thus provides a product having a smooth mouth feel and pleasing organoleptic properties.

In the direct method of tempering, the cocoa butter is heated to a temperature in excess of the highest polymorph melting temperature (i.e., to a temperature of 40° C. or higher). The chocolate is then cooled to allow chocolate crystals to form. In certain embodiments, the chocolate is cooled to approximately 27° C. for milk chocolate or approximately 29° C. for dark chocolate to allow forms IV and V to form. At this temperature, the chocolate is agitated to provide seed crystals to promote crystal formation. The chocolate is then typically heated to a temperature of at least approximately 31° C. to preferably eliminate type IV crystals and provide chocolate containing at least a significant fraction (and preferably nearly all) type V crystals. At this point the chocolate is considered “tempered” and may be, for example, poured into a mold of a desired shape and allowed to cool.

Another suitable tempering method utilized seed crystals. In this method, a portion of the chocolate desired to be tempered (e.g., two-thirds) is heated until the chocolate reaches its melting temperature. For dark chocolate, this temperature is approximately 49° C., while for milk chocolate this temperature is approximately 47° C. The remaining chocolate is then added and while stirring the following temperatures are reached: for dark chocolate 29° C. and for milk chocolate 27° C. The final step in the process is raising the temperature of the chocolate to its working temperature, which for dark chocolate is approximately 32° C. and for milk chocolate is approximately 30° C.

Addition of the EU extract (and optional mung bean extract) as additives, or inclusions has been observed to cause problems during the chocolate tempering process. Specifically, the presence of the active agent(s) has been observed to increase apparent viscosity not due to crystal growth in particular when the chocolate is cooled near the lowest cooling point prior to re-warming. In accordance with the present, programmed cooling is utilized to gradually slow down the cooling rate to encourage crystal growth. The optimum lowest cooling point is determined empirically to ensure optimum tempering is achieved.

Generally the melting temperature of certain dark chocolate compositions of the present invention including an EU extract or composition+a mung bean extract or composition is higher than that of an identical dark chocolate composition not containing the EU extract or composition and the mung bean extract or composition. Generally, the melting point of the EU+mung bean dark chocolate composition is greater than 30° C. Typically, the melting point of the EU+mung bean extract dark chocolate composition is greater than about 30.5° C., or greater than about 31° C. In certain embodiments, it has been discovered that incorporating the EU extract or composition and mung bean extract or composition increases the melting point of dark chocolate from approximately 30° C. to 31° C.

The viscosity of certain EU+mung bean dark chocolate compositions of the present invention (at 40° C.) is typically at least about 6000 centipoise (cps), at least about 7000 cps, at least about 8000 cps, or at least about 9000 cps. Thus, in certain embodiments the viscosity of certain EU+mung bean dark chocolate compositions of the present invention (at 40° C.) is from about 6000 to about 1400 cps, from about 8000 to about 1200 cps, or from about 9000 to about 1100 cps.

Generally, chocolate-based compositions of the present invention are formed into a suitable shape including, for example, square, rectangular, circular, spherical, or any other desired shape. Typically, chocolate-based compositions of the present invention are formed into a suitable shape and have a total weight of from about 5 to about 10 grams (g), or from about 5 to about 7.5 g. Generally, the composition has a chocolate content of at least about 70 wt %, at least about 80 wt %, or at least about 90 wt %.

The EU composition or extract generally constitutes at least about 1 wt %, at least about 1.5 wt %, or at least about 2 wt % of the composition. Typically, the EU composition or extract constitutes from about 0.5 to about 5 wt %, from about 1 to about 4 wt %, or from about 2 to about 3 wt % of the composition. Generally, the composition includes an EU composition extract loading of at least about 5 mg/g, at least about 10 mg/g, at least about 15 mg/g, or at least about 20 mg/g. Typically, the composition includes an EU extract loading of at least about 5 mg/g to about 40 mg/g, from about 10 mg/g to about 35 mg/g, from about 15 mg/g to about 30 mg/g, or from about 20 mg/g to about 30 mg/g.

When referring to a “EU composition or extract” concentration or loading it is to be understood that this refers to that composition or extract added to the chocolate-based composition. In various embodiments, a portion of an EU extract derived above may be utilized to provide a more concentrated extract that is ultimately utilized in the composition, for example, by drying such that only a portion of the initial extract is actually utilized in the composition. In this manner, an EU composition or extract loading of, for example, 150 mg may be referred to in terms of “extract ratio” indicating the total proportion of EU extract utilized to provide the extract portion introduced into the composition. For example, at an extract ratio of 10:1 and an EU composition or extract loading of 150 mg, approximately 1500 mg of an EU extract or composition is utilized to provide the EU composition or extract actually introduced into the chocolate-based composition. Similar considerations apply to mung bean extracts discussed below—for example, a proportion of mung bean extract introduced into a composition of 300 mg at an extract ratio of 10:1 indicates that a total 3000 mg of extract is utilized to provide the extract ultimately introduced into the composition.

Generally, a mung bean extract constitutes at least about 2 wt %, at least about 3 wt %, or at least about 4 wt % of the composition. Typically, the mung bean extract constitutes from about 2 to about 8 wt %, from about 3 to about 7 wt %, or from about 4 to about 6 wt % of the composition. Generally, the composition includes a mung bean extract loading of at least about 10 mg/g, at least about 20 mg/g, at least about 30 mg/g, or at least about 40 mg/g. Typically, the composition includes a mung bean extract loading of from about 10 mg/g to about 80 mg/g, from about 20 mg/g to about 70 mg/g, from about 30 mg/g to about 60 mg/g, or from about 40 mg/g to about 60 mg/g.

The compositions of the present invention generally include chocolate (e.g., milk chocolate or dark chocolate), and therefore include the ingredients of preparation thereof, specifically cocoa powder, sugar (cane and/or beet sugar), vanilla (typically natural extract), and chocolate liquor (typically alcohol free). Compositions of the present invention may also typically include soy lecithin (e.g., GMO-free (genetically modified organism-free) soy lecithin).

### Methods of Treatment

Compositions of the present invention are suitable for treatment of symptoms and conditions related to menopause. In particular, compositions of the present invention are suitable for treatment of various menopause-related symptoms and conditions, including one or more of the following: hot flushes, night sweats, palpations, irregular menstrual periods, cystitis/stress incontinence, vaginal dryness, vaginal irritation, thinning of skin and/or hair, headaches, loss of muscle tone, fatigue, joint pain, constipation or irritable bowel syndrome, periodontal (dental) disease, insomnia/sleeplessness, depression, anxiety, poor memory/concentration, decreased libido, and/or mood swings.

In certain embodiments, the methods of the present invention treat one or more of the following menopause-related symptoms or conditions: hot flashes, night sweats, headaches, fatigue, joint pain, insomnia/sleeplessness, anxiety, poor memory/concentration, and/or mood swings.

In certain embodiments, the methods of the present invention treat one or more of the following menopause-related symptoms or conditions: hot flashes, night sweats, fatigue, joint pain, and/or insomnia/sleeplessness.

Generally, methods of treatment of at least one menopause-related symptom or condition in accordance with the present invention comprise administration to a subject in need thereof of a composition of the present invention. Thus, typically such methods generally comprise administration of a chocolate-based composition of the present invention to a subject in need thereof.

The present invention is also directed to kits comprising one or more compositions of the present invention and instructions for their use, in particular directions to a subject for consuming one or more compositions of the present invention to treat one or more menopause-related symptoms or conditions.

In various embodiments, the methods of administration comprise administering to a subject 1 tablet twice daily, typically after consumption of food or as directed by a healthcare provider. Similarly, kits of the present invention typically comprise directions for the compositions of the present invention to be administered, or taken in this manner.

Having described the invention in detail, it will be apparent that modifications and variations are possible without departing from the scope of the invention defined in the appended claims.

### EXAMPLES

The following non-limiting examples are provided to further illustrate the present invention.

Examples 1-4 provide details regarding formulations tested and conditions for extraction of components thereof.

### Example 1

Eucommia ulmoides leaves (150 mg) (extracted at a 10:1 weight ratio using a (95:5) water:ethanol solvent extraction system)

Mung Bean (300 mg) (extracted at a 10:1 weight ratio using a (95:5) water:ethanol solvent extraction system)

5.5 grams of chocolate

RESULTS (4 women): not effective; no reduction in night sweats or hot flashes

### Example 2

Eucommia ulmoides leaves (100 mg) (extracted at a 10:1 weight ration using a (95:5) water:ethanol solvent extraction system)

Mung Bean (200 mg) (extracted at a 10:1 weight ratio using a(95:5) water:ethanol solvent extraction system)

100 mg of gamma-Aminobutyric acid

RESULTS (1 woman): not effective

### Example 3

Eucommia ulmoides leaves (150 mg) (extracted at a 10:1 weight ratio using a (95:5) water:ethanol solvent extraction system)

Mung Bean (300 mg) (extracted at a 10:1 weight ratio using a (95:5) water:ethanol solvent extraction system)

150 mg of Siberian ginseng (extracted at a 10:1 weight ratio using a (95:5) water:ethanol solvent extraction system)

RESULTS (1 woman): too bitter for compliance and chocolate is weak and deforms easily

### Example 4

Eucommia ulmoides Bark (150 mg) (extracted at a 10:1 weight ratio using a (20:80) water:ethanol solvent extraction system extraction)

Mung Bean (300 mg) (extracted at a 10:1 weight ratio using a (95:5) water:ethanol solvent extraction system)

5.5 grams of chocolate

RESULTS (13 women): surprisingly good results; 67% has positive results for hot flashes and 58% had positive results for night sweats

Individual results for each of the 13 subjects and also a summary of the survey are set forth in the following tables and graphs.

TABLE 1

| | | | | | What was your overall |
|---|---|---|---|---|---|
| | | Date | | | impression of the |
| | Time | Started | Week | What did you think of the | product (when taking |
| Subject | Submitted | Trial | Completed | concept? | active ingredients)? |
| 1 | Aug. 23, 2013 | May 29, 2013 | Jul. 17, 2013 | Liked it. | It reduced my hot |
| | 12:15 AM | | | | flashes |
| 2 | Aug. 22, 2013 | May 28, 2013 | Jul. 16, 2013 | I would have preferred pills as | It didn't work for me- |
| | 7:55 PM | | | opposed to chocolate because I | no change at all in |
| | | | | travel. | sympoms. |
| 3 | Aug. 20, 2013 | Jun. 28, 2013 | Aug. 23, 2013 | I love this product, it's easy to | Great product, taken |
| | 12:14 PM | | | take, tastes great and actually | twice daily I always |
| | | | | works! | take it after a meal, so |
| | | | | | as to get that little |
| | | | | | sweet nibble we all |
| | | | | | crave. Easy, tasty and |
| | | | | | it has taken my |
| | | | | | symptoms down to a |
| | | | | | bare minimum I will |
| | | | | | buy this product! |
| 4 | Aug. 20, 2013 | May 27, 2013 | Jul. 23, 2013 | I was pleased with the | I believe it had a |
| | 8:40 AM | | | product, especially when I | positive effect, easy to |
| | | | | stopped and felt the symptoms | take, and worth |
| | | | | return. | continuing. |
| 5 | Aug. 20, 2013 | May 29, 2013 | Jul. 24, 2013 | good concept-easy way to take | good taste, |
| | 12:32 AM | | | a supplement | |
| 6 | Aug. 19, 2013 | | | Like the food (dessert) format. | Dramatically reduced |
| | 8:35 PM | | | | my hot flashes. Didn't |
| | | | | | really notice other |
| | | | | | symptoms, except my |
| | | | | | energy levels did |
| | | | | | improve with the |
| | | | | | multi. |
| 7 | Aug. 19, 2013 | May 24, 2013 | Jul. 20, 2013 | Good concept for som, but I | Did not really see an |
| | 7:43 PM | | | didn't like eating chocolate | improvement. |
| | | | | everyday. | |
| 8 | Aug. 19, 2013 | Jun. 2, 2013 | Jul. 28, 2013 | Most women love chocolate, so | Definite reduction of |
| | 7:10 PM | | | to have a daily dose of healthy | hot flashes, |
| | | | | chocolate with ingredients that | sleeplessness and night |
| | | | | help to minimise the symptoms | sweats. However, I |
| | | | | of menopause, is a fun, unique | think I would have |
| | | | | concept. It's like having a treat | needed a higher dosage |
| | | | | instead of popping a pill. | to be really effective. |
| 9 | Aug. 19, 2013 | Jun. 3, 2013 | Jul. 28, 2013 | | |
| | 4:55 PM | | | | |
| 10 | Aug. 19, 2013 | May 23, 2013 | Jul. 25, 2013 | Love the product | Good effects |
| | 4:52 PM | | | | |
| 11 | Aug. 19, 2013 | May 29, 2013 | Aug. 14, 2013 | i love love love the chocolate | My blood pressure is |
| | 3:09 PM | | | | 118/80,,, perfect. It |
| | | | | | had made an incredible |
| | | | | | difference in the joint |
| | | | | | pain my hips, knees |
| | | | | | and ankles). |
| 12 | Aug. 19, 2013 | | | I liked it | I did not think it helped |
| | 12:47 PM | | | | much at the time, but |
| | | | | | am willing to keep |
| | | | | | trying for longer term. |
| 13 | Aug. 19, 2013 | May 29, 2013 | Jul. 31, 2013 | The chocolate is a nice change | I thought it really |
| | 11:26 AM | | | from pills | helped with hot flashes |
| | | | | | and/or feeling warm in |
| | | | | | general |

TABLE 2

| | What do you | | |
|---|---|---|---|
| | think of the | | Any final comments related to |
| | taste by end of | | chocolate format? Ease of Use, |
| Subject | trial? | Comments on taste | compliance, etc? |
| 1 | I'm indifferent | | Not fond of dark chocolate |
| 2 | I'm indifferent | I didn't like it at all at first but got | |
| | | used to it. | |
| 3 | I like | sweet, tastes great. | Easy to use, quick tasty and simple. |
| 4 | I like | | Little hard to travel with, not compact, |
| | | | and melted easilt in heat. |
| 5 | I like | no problem-tasted great | enjoyable to take twice a day likely |
| | | | don't need the sugar |
| 6 | I like | | |
| 7 | I'm indifferent | | As mentioned did not like eating |
| | | | chocolate everyday, two times a day. |
| | | | Not easy to travel with re melting. |
| 8 | I like | I love bitter chocolate, and I'm sure | Wafers are ‘classy’ . . . and easier to |
| | | the active ingredients made it more | consume than a chunk of chocolate or |
| | | bitter. But to have that bitter taste | something chewy . . . especially since |
| | | right after my meals, wasn't the | we're supposed to take it after a meal. |
| | | ideal effect on my tastebuds. | |
| 9 | I'm indifferent | | I prefer less chocolate and smaller |
| | | | size |
| 10 | I like | | Just have to brush teeth |
| 11 | I like | fabulous | I hope you keep the chocolate format |
| 12 | I like | | |
| 13 | I like | It tastes good, it's fine. | Nice treat to have chocolate each day:) |

TABLE 3

| | Menopause Symptoms Score Sheet Please indicate Improvement | | | | | |
|---|---|---|---|---|---|---|
| | while on active ingredient only (ie first 4 weeks) | | | | | |
| | | | | | Cystitis/ | |
| | | | | Irregular | stress | Vaginal |
| Subject | Hot flushes | Night sweats | Palpitations | periods | incontinence | dryness |
| 1 | Improvement | Improvement | Improvement | NA | NA | NA |
| 2 | No Improvement | No Improvement | NA | NA | NA | No Improvement |
| 3 | Improvement | Improvement | NA | Improvement | NA | NA |
| 4 | Improvement | Improvement | Improvement | NA | NA | Improvement |
| 5 | Improvement | Improvement | NA | NA | NA | NA |
| 6 | Improvement | NA | NA | NA | NA | No Improvement |
| 7 | | | | | | |
| 8 | Improvement | Improvement | NA | NA | NA | NA |
| 9 | NA | Improvement | Improvement | NA | Improvement | Improvement |
| 10 | Improvement | NA | NA | NA | NA | Improvement |
| 11 | NA | NA | NA | NA | NA | NA |
| 12 | No Improvement | No Improvement | NA | NA | Improvement | NA |
| 13 | Improvement | Improvement | NA | NA | NA | No Improvement |

TABLE 4

| | Menopause Symptoms Score Sheet Please indicate Improvement | | | | | | |
|---|---|---|---|---|---|---|---|
| | while on active ingredient only (ie first 4 weeks) | | | | | | |
| | | | Thinner skin | | Loss of | | |
| Subject | Vaginal irritation | Weight gain | and hair | Headaches | muscle tone | Fatigue | Joint pain |
| 1 | NA | NA | No Improvement | NA | No Improvement | NA | NA |
| 2 | NA | No Improvement | NA | NA | No Improvement | No Improvement | NA |
| 3 | No Improvement | No Improvement | NA | Improvement | NA | Improvement | Improvement |
| 4 | Improvement | NA | Improvement | Improvement | NA | Improvement | Improvement |
| 5 | NA | NA | NA | NA | NA | NA | NA |
| 6 | NA | No Improvement | NA | NA | NA | Improvement | NA |
| 7 | | | | | | | |
| 8 | NA | No Improvement | NA | NA | No Improvement | NA | NA |
| 9 | Improvement | No Improvement | NA | Improvement | Improvement | Improvement | Improvement |
| 10 | Improvement | NA | No Improvement | Improvement | NA | Improvement | Improvement |
| 11 | NA | No Improvement | NA | NA | NA | Improvement | Improvement |
| 12 | NA | No Improvement | NA | NA | No Improvement | No Improvement | NA |
| 13 | NA | NA | No Improvement | NA | NA | Improvement | Improvement |

TABLE 5

| | Menopause Symptoms Score Sheet Please indicate Improvement while on active | | | | |
|---|---|---|---|---|---|
| | ingredient only (ie first 4 weeks) | | | | |
| | Constipation or | | | | |
| | Irritable Bowel | Periodontal | Insomnia/ | | |
| Subject | Syndrome | (dental) disease | Sleeplessness | Depression | Anxiety |
| 1 | NA | NA | Improvement | NA | NA |
| 2 | NA | NA | No Improvement | NA | NA |
| 3 | NA | NA | Improvement | Improvement | Improvement |
| 4 | Improvement | NA | Improvement | No Improvement | Improvement |
| 5 | NA | NA | NA | NA | NA |
| 6 | NA | NA | No Improvement | NA | NA |
| 7 | | | | | |
| 8 | NA | NA | Improvement | No Improvement | No Improvement |
| 9 | NA | NA | Improvement | Improvement | Improvement |
| 10 | NA | Improvement | Improvement | NA | Improvement |
| 11 | NA | NA | No Improvement | NA | NA |
| 12 | NA | NA | No Improvement | No Improvement | NA |
| 13 | NA | NA | Improvement | Improvement | Improvement |

TABLE 6

| | Menopause Symptoms Score Sheet Please indicate | | | |
|---|---|---|---|---|
| | Improvement while on active ingredient only | | | |
| | (ie first 4 weeks) | | | |
| | Poor memory/ | Decreased | | Any “side efffects” that stood |
| Subject | concentration | libido | Mood swings | out for you |
| 1 | Improvement | NA | NA | |
| 2 | No Improvement | NA | NA | no |
| 3 | Improvement | Improvement | Improvement | none. |
| 4 | Improvement | No Improvement | Improvement | no |
| 5 | NA | NA | NA | none |
| 6 | No Improvement | NA | NA | Not that I'm aware of |
| 7 | | | | Adult acne returned and IBS flared up. |
| 8 | No Improvement | No Improvement | No Improvement | No |
| 9 | NA | NA | Improvement | |
| 10 | NA | Improvement | Improvement | |
| 11 | Improvement | NA | NA | I did get a vaginal infection during the |
| | | | | last 2 weeks of trial . . . I dont think it |
| | | | | was related to the product . . . I also had |
| | | | | a mild case of diverticulities |
| 12 | No Improvement | NA | No Improvement | |
| 13 | No Improvement | NA | NA | no side effects noticed |

### Example 5

This Example provides compositional details for 90 kg batch and individual 6 q tablets of the present invention.

| INGREDIENT | 90 kg Batch | 6 g tablet | | % |
|---|---|---|---|---|
| Eucommia | 2.25 kg | 150 | mg | 2.50% |
| Mung Bean Extract | 4.5 kg | 300 | mg | 5.00% |
| Dark Chocolate (72% Cocoa) | 83.25 kg | 5.55 | g | 92.50% |
| TOTAL | 90.00 kg | 6 | g | 100.00% |

### Example 6

This Example provides compositional details for chocolate-based tablets in accordance with the present invention.

1 square tablet:

Medicinal Ingredients: Mung-bean (Vigna radiate var. radiata, seed) 300 mg (10:1 extract equivalent to 3000 mg); Eucommia (Eucommia ulmoides, branch bark) 150 mg (10:1 extract equivalent to 1500 mg).

Non-Medicinal ingredients: Cocoa powder, cane and beet sugar, GMO-free soy lecithin, natural vanilla extract, chocolate liquor (alcohol free).

When introducing elements of the present invention or the preferred embodiments(s) thereof, the articles “a”, “an”, “the” and “said” are intended to mean that there are one or more of the elements. The terms “comprising”, “including” and “having” are intended to be inclusive and mean that there may be additional elements other than the listed elements.

In view of the above, it will be seen that the several objects of the invention are achieved and other advantageous results attained.

As various changes could be made in the above compositions and methods without departing from the scope of the invention, it is intended that all matter contained in the description herein shall be interpreted as illustrative and not in a limiting sense.

1. An edible chocolate composition comprising at least one pharmaceutically or nutraceutically active agent, the composition comprising:
a chocolate base, an eucommia ulmoides (EU) composition, and a mung bean composition, wherein the EU composition and the mung bean composition are dispersed throughout the chocolate base.

2. The composition of claim 1, wherein the edible composition is a solid composition.

3. The composition of claim 1, wherein the chocolate base is selected from the group consisting of sweet chocolate, semi-sweet chocolate, dark chocolate, milk chocolate, white chocolate, couverture chocolate, baking chocolate, and combinations thereof.

4. (canceled)

5. (canceled)

6. The composition of claim 1, wherein the melting temperature is greater than 30° C.

7. The composition of claim 6, wherein the viscosity of the composition (at 40° C.) is at least about 6000 centipoise (cps).

8. The composition of claim 6, wherein the viscosity (at 40° C.) is from about 6000 to about 1400 cps.

9. The composition of claim 1, wherein the composition has a chocolate content of at least about 70 wt %.

10. The composition of claim 1, wherein the EU composition is an EU extract derived from leaves and/or bark of a plant and, prior to dispersing throughout the chocolate base, has a solids content of at least about 70 wt %.

11. The composition of claim 10, wherein the EU extract constitutes at least about 1 wt %.

12. The composition of claim 10, wherein the EU extract constitutes from about 0.5 to about 5 wt %.

13. The composition of claim 10, wherein the EU extract is present at a loading of at least about 5 mg/g composition.

14. (canceled)

15. The composition of claim 1, wherein the EU extract comprises one or more compounds selected from the group consisting of caffeic acid, chlorogenic acid, ferulic acid, quercetin, rutin, isoquercitrin, ulmoidol, corosolic acid, oleanolic acid, ursolic acid, foliasalacioside, quercetin, lignan diglucosides, pinoresinol, glucopyranoside, syringaresinol, gutta-percha, glucans, eucommioside, eucommiol, geniposide, ulmoprenol, and combinations thereof.

16. The composition of claim 1, wherein the EU extract is derived from leaves and comprises one or more compounds selected from the group consisting of caffeic acid, chlorogenic acid, ferulic acid, quercetin, rutin, isoquercitrin, ulmoidol, corosolic acid, oleanolic acid, ursolic acid, foliasalacioside, quercetin, and combinations thereof.

17. The composition of claim 1, wherein the EU extract is derived from bark and comprises one or more compounds selected from the group consisting of lignan diglucosides, pinoresinol, glucopyranoside, syringaresinol, gutta-percha, glucans, eucommioside, eucommiol, geniposide, ulmoprenol, and combinations thereof.

18. The composition of claim 1, wherein the mung bean composition is a mung bean extract which, prior to dispersing throughout the chocolate base, has a solids content of at least about 70 wt %.

19. The composition of claim 18, wherein the mung bean extract constitutes at least about 2 wt %.

20. (canceled)

21. The composition of claim 18, wherein the mung bean extract is present at a loading of at least about 10 mg/g composition.

22. (canceled)

23. The composition of claim 1, wherein the composition further comprises soy lecithin, one or more flavoring agents, and/or chocolate liquor.

24. A method of treatment of one or more menopause-related symptoms or conditions, the method comprising administering to a subject in need thereof a composition as defined in claim 1.

25. The method of claim 24, wherein the one or more menopause-related symptoms or conditions are selected from the group consisting of hot flashes, night sweats, palpations, irregular menstrual periods, cystitis/stress incontinence, vaginal dryness, vaginal irritation, thinning of skin and/or hair, headaches, loss of muscle tone, fatigue, joint pain, constipation or irritable bowel syndrome, periodontal (dental) disease, insomnia/sleeplessness, depression, anxiety, poor memory/concentration, decreased libido, mood swings, and combinations thereof.

26. (canceled)

27. (canceled)
